(19) United States
(12) Patent Application Publication
(10) Pub. No.: US 2021/0293707 A1
(43) Pub. Date: Sep. 23, 2021

(54) Fluorescence Lifetime Well Array Reader and Actuator

(71) Applicant: The Charles Stark Draper Laboratory, Inc., Cambridge, MA (US)

(72) Inventors: Ian W. Frank, Arlington, MA (US); Andrew P. Magyar, Arlington, MA (US); Cory Larsen, Cambridge, MA (US); Jonathan S. Ung, Brighton, MA (US); Kasey J. Russell, Cambridge, MA (US); Kirsty A. McFarland, Melrose, MA (US); Jeffrey A. Korn, Lexington, MA (US)

(21) Appl. No.: 17/265,456

(22) PCT Filed: Aug. 2, 2019
(86) PCT No.: PCT/US2019/044827

§ 371 Date: Feb. 2, 2021

Related U.S. Application Data
(60) Provisional application No. 62/713,807, filed on Aug. 2, 2018.

(51) Int. Cl. G01N 21/64; C12Q 1/686; B01L 3/00

(52) U.S. Cl. CPC G01N 21/6408; G01N 21/6454; G01N 21/6486; B01L 2300/0832; B01L 3/5085; B01L 2300/0829; C12Q 1/686

(57) ABSTRACT

A well reader and actuator (100) for a well array (50) has an array of detectors, each detector for detecting a fluorescence signal from fluorophores in a respective well of the well array and an excitation subsystem for exciting the fluorophores in the wells of the well array. Embodiments of this invention can be used to carry out two important functions in a highly parallel manner: by addressing individual wells in a 32, 96, 384 etc. well array, where each well contains a potential chemical or biological reaction. The two functions are: 1) through thermal, optical or other means and combinations thereof the rate of a chemical or biological reaction is controlled or gated (e.g. colder wells inhibit a reaction, or an enzymatic reaction requires blue light to proceed); and 2) through use of fluorescent species that are sensitive to the target reaction—or reactions—an optical readout of fluorescent intensity and/or lifetime is tracked to monitor the evolution of the reaction.

### RELATED APPLICATIONS

This application claims the benefit under 35 USC 119(e) of U.S. Provisional Application No. 62/713,807, filed on Aug. 2, 2018, which is incorporated herein by reference in its entirety.

### BACKGROUND OF THE INVENTION

Well arrays are used as large sets of small test tubes in analytical research and clinical diagnostic testing. A common example of a well array is a microplate or microwell plate. These are flat plates with multiple wells. Another example is “Eppendorf” or microcentrifuge tube rack. Here, a set of tubes are held in a rack for convenience and running a large number of separate tests. Well arrays also come in various sizes. The most common well array format is the 96-well array in an 8 by 12 matrix. Larger arrays such as 384- or 1536-well arrays are also available.

Plate readers are instruments that are used to detect biological, chemical or physical events associated with samples in the well arrays. They are widely used in research, drug discovery, bioassay validation, quality control and manufacturing processes in the pharmaceutical and biotechnological industry and academic organizations.

A common detection mode for plate readers is fluorescence intensity detection. In these readers, a light source illuminates the samples in the wells of the well arrays at a wavelength sufficient to excite fluorophore of interest in the samples. The fluorescence light from the samples is then detected and characterized.

Another detection mode is time-resolved or lifetime fluorescence detection. Historically this has employed lanthanides fluorophores that have longer fluorescence lifetimes. Here, the detection system detects the fluorescence light after operation of the light source.

At the same time, PCR is a powerful technique for amplifying short sections of DNA. With PCR, one can quickly produce millions of copies of DNA starting from a single template DNA molecule. PCR includes a three phase temperature cycle of denaturation of DNA into single strands, annealing of primers to the denatured strands, and extension of the primers by a thermostable DNA polymerase enzyme. For example, the first stage, at around 95° C., allows the separation of the nucleic acid's double chain. The second stage, at a temperature of around 50-60° C., allows the binding of the primers with the DNA template. The third stage , at between 68-72° C., facilitates the polymerization carried out by the DNA polymerase. This three stage cycle is repeated so that there are enough copies to be detected and analyzed. In principle, each cycle of PCR could double the number of copies. In practice, the multiplication achieved after each cycle is always less than 2. Furthermore, as PCR. cycling continues, the buildup of amplified DNA products eventually ceases as the concentrations of required reactants diminish.

Real-time PCR or quantitative Polymerase Chain Reaction (qPCR) refers to measuring the buildup of amplified DNA products as the reaction progresses, typically once per PCR cycle. Monitoring the accumulation of products over time allows one to determine the efficiency of the reaction, as well as to estimate the initial concentration of DNA template molecules.

### SUMMARY OF THE INVENTION

Embodiments of this invention can be used to carry out two important functions in a highly parallel manner: by addressing individual wells in a 32, 96, 384 etc. well array, where each well contains a potential chemical or biological reaction. The two functions are: 1) through thermal, optical or other means and combinations thereof the rate of a chemical or biological reaction is controlled or gated (e.g. colder wells inhibit a reaction, or an enzymatic reaction requires blue light to proceed); and 2) through use of fluorescent species that are sensitive to the target reaction—or reactions—an optical readout of fluorescent intensity and/or lifetime is tracked to monitor the evolution of the reaction.

One possible function to be performed and monitored in the wells is quantitative Polymerase Chain Reaction (PCR). Here, the goal is to monitor the rate at which an enzymatic reaction is able to copy and amplify species of DNA or the enzymatic reaction could extend chains of DNA by adding nucleotides or raw base pairs. The reaction is dependent on, among other things, temperature cycles. Therefore, an effective qPCR machine will be able to cycle the temperature of the reaction wells in a specified manner. In the case of a PCR the reaction is monitored through the use of an intercolation dye. The dye has the property of changing its fluorescent intensity and/or fluorescent lifetime as more DNA is produced and the dye binds to the DNA. This means an effective qPCR machine can illuminate the reaction well with light of a wavelength that will be absorbed by the fluorescent dye and be able to collect and quantify the light emitted at the longer wavelength as the fluorescent molecule relaxes back to a lower energy.

Today qPCRs do not carry out the temperature cycling or the fluorescent interrogation on a per-well basis, instead they control the temperature across an entire well plate, and uniformly illuminate and image the fluorescence over the entire well plate. They also do not allow for multiple wavelengths of light to be targeted at the wells on a per-well basis, with illumination patterns controlled on a per well basis. This can be valuable to, for example, control the qPCR reaction enzyme, or alternatively for implementing other assays designed to screen enzymes or other biomolecules for photo-activity. Enzymes that have been modified to only become active under illumination from one wavelength (activation-wavelength), typically a different wavelength than the wavelength required to excite the fluorescent monitor (exciting-wavelength), typically different from a third wavelength or wavelength range that the dye will emit (emitting-wavelength(s)). Per well control of such an enzymatic reaction would require not only the temperature cycling but also illumination by the excitation-wavelength at varying intensities on a per-well basis. In an alternative implementation, multiplexed fluorescent monitors can be used to characterize PCR associated with different probes. In this implementation, two or more exciting-wavelengths and two or more emitting-wavelengths are required.

qPCR analysis now focuses on the total intensity of the fluorescence emitted from the reaction well. However, the increased or decreased fluorescent intensity is almost always accompanied by a significant change in the radiative lifetime of the fluorescent dye. This gives a potential second avenue for following the reaction in question. Many qPCRs do not use this method because the lifetimes of the dyes in use are in the 1-10 nanosecond (ns) range so switching rates required to effectively monitor this property of the dye need to be in the MegaHertz (MHz) to GigaHertz (GHz) regime.

The present system provides a platform where this modulation can occur at the rates required with the close integration of the electronics in combination with the use of fast diodes, such as light emitting diodes (LEDs) or laser diodes, and fast photodiodes instead of lamps and cooled CCD cameras, for example.

Each of the individual wells in e.g. a 96 well plate can be illuminated with different colors of light at different times. This allows tracking of multiple types of intercalating dyes or dye-quencher pairs in the same measurements, as well as enabling the apparatus to examine the effects of chromatically sensitive illumination during growth cycles.

This invention also enables chemical and biological reactions to be monitored for species concentration, such as the oxygen levels inside a fluid. This is accomplished through the use of special dyes that are sensitive to the concentration of e.g. oxygen, and by monitoring the intensity or relative phase of the dye under a modulated excitation.

Additionally, the use of fast electronics and light emitting diodes or laser diodes means that the excitation light can be modulated allowing the readout signal to be demodulated using a lock-in amplifier (on a per-well basis with the modulation frequencies also provided on a per well basis). This can be used to eliminate leakage into the readout signal of common noise sources such as 60 Hz AC flickering illumination from fluorescent light bulbs, and other electronic noise sources. It can be used to reduce or eliminate cross-talk between wells. It further allows for phase sensitive measurements in which a measurement of the properties of the fluorescent molecules is independent of the total intensity and therefore less susceptible to bleaching. Also, the modulation frequencies can be optimally located based on the difference in fluorescent lifetimes between two different states, e.g. for an intercalation dye and qPCR, the dye has a characteristic intensity and a characteristic lifetime that will change as the dye binds to oligonucleotides. The ideal modulation frequency will maximize the phase contrast between the two states, which is often the inverse of the harmonic mean of the two characteristic lifetimes. For many dyes with characteristic lifetimes in the picosecond to nanosecond to microsecond ranges, this frequency is 10 s to 100 s of GHz, but is sometimes less, with such frequencies in the MHz or even kilohertz (kHz). All of these frequencies are reachable with well-designed proximal electronics and diodes but would be difficult to achieve with traditional lamp-based qPCR systems.

In general, according to one aspect, the invention features a well reader for a well array. It comprises an array of detectors, each detector for detecting a fluorescence signal from fluorophores in a respective well of the well array, and an excitation subsystem for exciting the fluorophores in the wells of the well array.

Typically, the well array has 96 wells. Those wells could be an array of microcentrifuge tube or a well array substrate having an array of wells.

In the current embodiment, the reader further comprises a printed circuit board and optical block for interfacing the printed circuit board to the well array. This printed circuit board could include high-speed drivers and analog to digital conversion circuits, along with possibly a controller for performing a convolution between a drive signal to the excitation subsystem and a signal produced by the detectors. Sometimes, multiplexer is useful between the array of detectors and the analog to digital conversion circuits to lower cost.

In the typically application, the well reader assesses changes in a radiative lifetime of one or more fluorophores in the well array.

In addition, the array of detectors can include multiple photodiodes for each of the wells.

The excitation subsystem could be implemented as a light source for each of the wells. And, that light source might include multiple diodes for interrogating and/or heating samples held in the respective well of the well array.

In general, according to another aspect, the invention features a method of operation of a well reader. This method comprises exciting fluorophores in wells of the well array and detecting a fluorescence signal from one or more fluorophores in the wells of the well array with an array of detectors.

In general, according to another aspect, the invention features a well reader and actuator for a well array. This reader comprises an array of unit well subsystems for the well array and a controller for controlling monitoring polymerase chain reactions in the wells via the unit well subsystems.

In general, according to another aspect, the invention features a method of DNA quantification that uses optical phase modulation to measure changes in fluorescence lifetime of DNA binding dyes during polymerase chain reactions taking place in a well array.

In general, according to another aspect, the invention features a reaction system, comprising a well array and a fluorescence lifetime analysis system for assessing changes in the radiative lifetime of fluorophores in the well array.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views, The drawings are not necessarily to scale; emphasis has instead been placed upon illustrating the principles of the invention. Of the drawings:

FIG. 1 is a perspective view of an integrated reader and actuator for a tube-based well array, which has been constructed according to the principles of the present invention.

FIG. 2 is a perspective cross-sectional view of the reader and actuator.

FIG. 3 is a side cross-sectional view of a single well and the respective unit well subsystem of the reader and actuator.

FIG. 4 is a perspective view of an integrated reader and actuator for a well-plate-based well array, according to another embodiment.

FIG. 5 is a perspective cross-sectional view of the second embodiment of the reader and actuator.

FIG. 6 is a side cross-sectional view of a single well and the respective unit well subsystem of the reader and actuator of the second embodiment.

FIG. 7A is a perspective view showing the details of the upper subblock; and FIG. 7B is a cross section view of a portion of the upper subblock.

FIG. 8A is a perspective view showing the details the middle subblock; and FIG. 813 is a cross section view of a portion of the middle subblock.

FIG. 9A is a perspective view showing the details the lower subblock; and FIG. 9B is a cross section view of a portion of the lower subblock.

FIG. 10A is a perspective view of a skirt that aligns the optical block to the tube frame; and FIG. 10B is a cross sectional view showing the skirt in relation to the optical block and well substrate.

FIG. 11 is a block diagram of the controller and its connection to the unit well subsystems and high-speed drivers and analog to digital conversion circuits 132.

FIG. 12 is a block diagram showing the one implementation of a light source of the reader.

FIG. 13 is a block diagram showing the one implementation of a light detector of the reader.

FIG. 14 is a cross section view of the optical block according to another embodiment.

FIG. 15 plot of signal intensity as function of time in minutes for fresh Tdt, Tdt on ice, qPCR sample with no enzyme, a sample with no enzyme on ice and qPCR with Tdt.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may he present.

Unless otherwise defined, all terms (including technical and scientific terms) used herein have the same meaning as commonly understood by one of ordinary skill in the art to which this invention belongs. it will be further understood that terms, such as those defined in commonly used dictionaries, should be interpreted as having a meaning that is consistent with their meaning in the context of the relevant art and will not be interpreted in an idealized or overly formal sense unless expressly so defined herein.

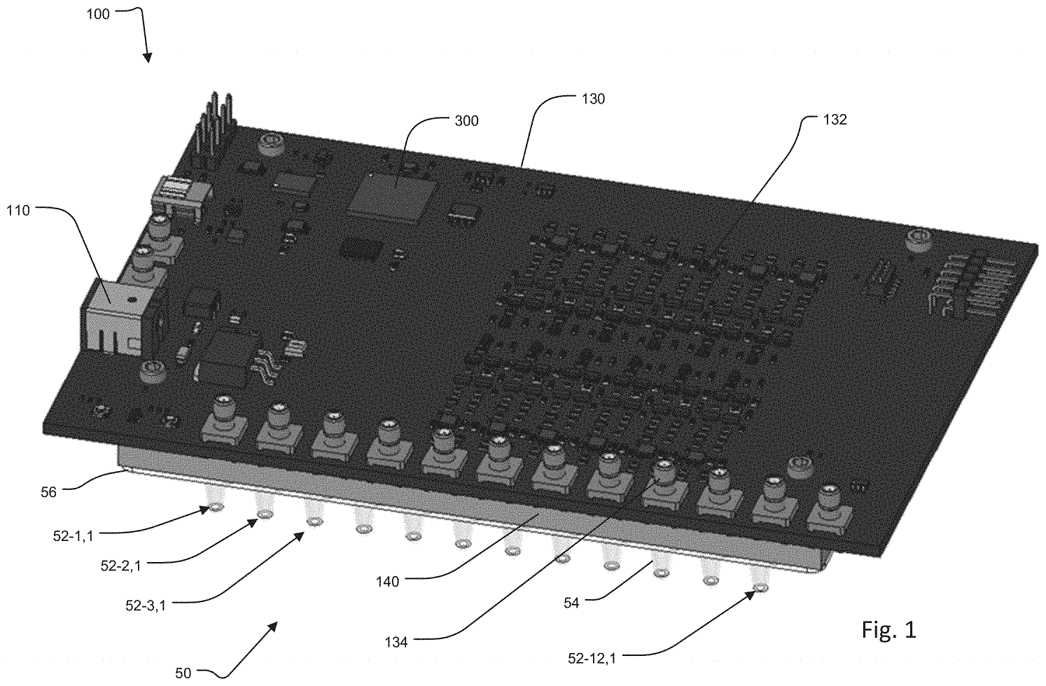

FIG. 1 is a perspective view of the well array 50 with an integrated fluorescence lifetime reader and actuator 100 attached to the well array 50, which reader has been constructed according to the principles of the present invention. This yields a chemical reaction system in which chemical reactions in the well array 50 are monitored and possibly controlled by the reader and actuator 100.

In general, the reader 100, has an array of unit well subsystems 142 wherein each unit well subsystem is miniaturized to operate adjacent to a respective well 52 of the well array 50. The example reader 100 accommodates 96 wells in a 12×8 well array. But readers for larger or smaller well arrays are of course possible and within the scope of this invention.

In the illustrated embodiment, the reader 100 includes a printed circuit board (PCB) 130 that functions as an electronic motherboard for the reader 100. It also functions as a mechanical frame that supports the reader.

On the PCB 130, there are high-speed drivers and analog to digital conversion circuits 132 and a controller 300. Also included as part of the PCB 130 are the electronics for communications for control and readout of data from the reader 100. In the illustrated embodiment, the PCB 130 thus further includes a data interface 110, such as a network or bus interface. In one embodiment, this is an Ethernet (145) or universal serial bus (USB) jack that may also provide power to the PCB 130 such as using the power over ethernet (POE) or USB power delivery protocol.

Also included on the PCB 130, in the particular embodiment illustrated, are a series of SMC (SubMiniature version C) connectors 134, which are coaxial radio frequency (RF) connectors. These are used to monitor the operation of the electronics on the PCB 130.

On the distal side of the PCB 130 is an optical block 140. This optical block provides the optical interface between the electronics of the PCB 130 and wells 52 of the well array 50. In the illustrated embodiment, the wells 52 are implemented as a set of Eppendorf or microcentrifuge tubes 54 held together in the well array 50 by a tube frame 56. Note that in the figures, the position in the array of wells of individual units is indicated by their x, y Cartesian coordinates, so well 52-2,1 designates the well in the second column of the first row.

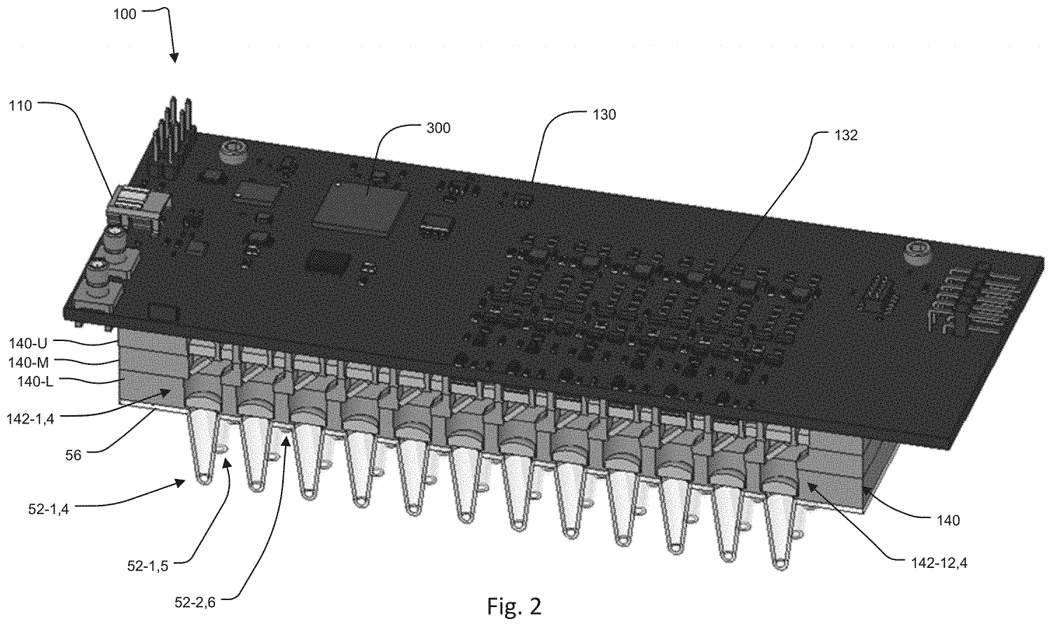

FIG. 2 shows the relationship between the optical block 140, each of the wells 52 of the well array 50 and the unit well subsystems 142 of the optical block 140 that are associated with each well 52.

Specifically, the optical block 140 is arranged in an array of unit well subsystems 142-x,y. The illustrated example has 96 such subsystems. Each subsystem of the block 140 comprises electro-optical components, which are driven and monitored by the high-speed driver and analog-to-digital circuits 132 of the PCB 130, and further includes optical components such as lenses and filters that allow the coupling of light into each respective well and detection of light from each of the wells 52x,y.

In the illustrated example, the optical block 140 comprises a stack of subblocks for holding the components: specifically, an upper block 140-U, a middle block 140-M, and a lower block 140-L.

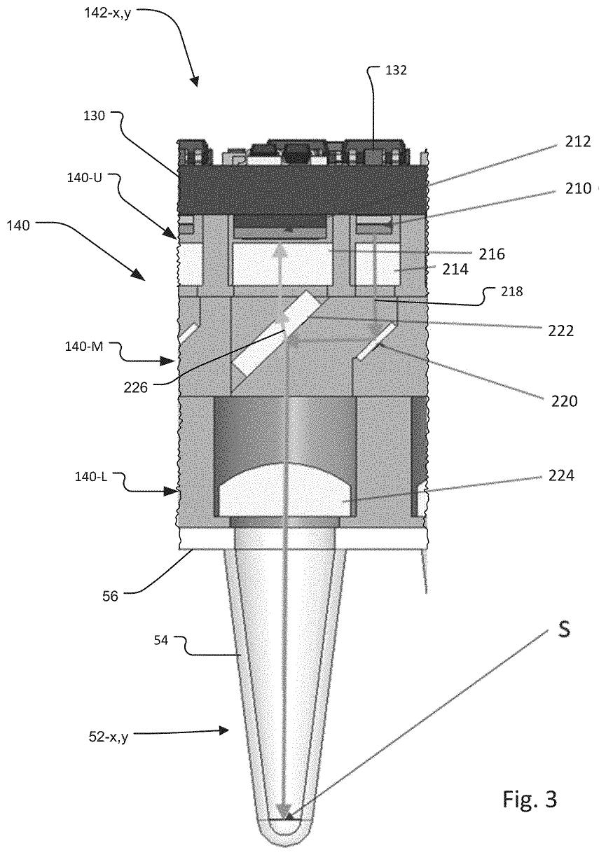

FIG. 3 is a side plan view of an exemplary single well 52-x,y and respective the unit well subsystem 142-x,y associated with the well 52.

In general, each single well 52 of the reader 100 has its own optical and optoelectronic components for generating light that is transmitted into the well and detecting light from the well. In the illustrated example, the sample S of the well 52 is contained in a microcentrifuge Eppendorf tube 54, such as a tube holding about 0.2 milliliters (mL) of fluid or less. The tube 54 is then secured in its array by an integral or separate tube frame 56. The unit well subsystem 142 includes a light source 210. Also included are additional optoelectronic components such as a light detector 212, e.g., one or more photodiodes, that then detect the light returning from the tube 54 after it is been modulated or otherwise changed by the sample S contained in the well 52 of the tube 54.

This side view also shows the construction of the optical block 140. Specifically, the optical block 140 comprises the upper subblock 140-U, which mainly houses the optoelectronic components such as the LED or laser diodes of the light source 210 and the photodiodes or other sensors of the light detector 212. The middle subblock 140-M holds optical components that facilitate the coupling and filtering of light to and from the well 52. Finally, the lower subblock 140-L contains optical elements such as lenses and in the typical embodiment seals against the top mouth of each tube 54.

In more detail, in the preferred embodiment, the light for interrogating the sample is generated by the light source 210. The interrogation light is typically generated by a LED or laser diode. In some embodiments, the LED or laser diode is tunable so that it can generate light at different wavelengths. In other embodiments, the light source 210 comprises several LEDs and/or laser diodes, such as several diodes that each generate light at different wavelengths. In some embodiments, the light source 210 may even further generate white light. In one specific example, the light source 210 is part of an excitation subsystem that includes one or more LEDs or laser diodes for each well that generates light at wavelengths for exciting fluorophores in the sample S along with at least one addition heating LED or laser diode that generates light that will be absorbed by the sample S or the material defining the well (tube 54) to enable the optical heating of the sample S. Typically heating diodes generate light in the infrared wavelengths to thereby control the temperature of the sample. In this way, the temperature of each well can be individually controlled under the control of the controller 300 of the PCB 130.

The light 218 generated by the light source 210 passing through a source filter 214. This source filter 214 is designed to transmit the one or more wavelengths that are generated by the light source 210. This source light is then reflected by a mirror 220, angled at 45° with respect to the axis of the source light 218, to a dichroic filter 222.

The dichroic filter 222 is also angled at 45° with respect to the optical axis of the source light 218 from the light source 210. Further, the dichroic filter is designed to reflect light at the wavelengths generated by the light source 142. Thus the light is reflected downward through a focusing lens 224, e.g., planoconvex, so that it is focused on the sample S held in the bottom of the tube 54.

The light from the light source 210, in one mode of operation, heats the sample S, such as to a temperature at which the desired reaction can take place. In other modes of operation, the source light 218 from the light source 210 will be modulated by the sample S. In some examples, the source light 218 might be absorbed by the sample and then re-irradiated as a fluorescence or Raman signal.

This sample modulated light 226 from the sample S is collected by the lens 224. The modulated light 226 is directed to the dichroic filter 222.

The dichroic filter 222 is designed to pass the wavelengths of light that are expected from the sample such as light at the emitting-wavelength(s) of the fluorophore. This light is transmitted through a detector filter 216 and then detected by the light detector 212 such as a one or more detection elements such as photodiodes or microbolometers. In some embodiments, there may be several photodiodes that are sensitive to light at different wavelengths. Moreover, the temperature of the sample could be further resolved by detecting blackbody radiation such as with the microbolometer of the light detector 212. Closed loop feedback between a microbolometer and a heat source can provide precise control of the temperature on a per-well basis. To enable multiplexed collection the detector filter 216 and/or the dichroic filter 222. have multiple passbands in some examples to transmit fluorescence photons as well as thermal photons. In other cases, multipart filters are used, each for a different detection element of the light detector 212. In some cases, the dichroic filter is replaced by a half silvered minor, and filtering is carried out solely at the light detector 212. In other instances, a grating or prism is used to direct emitting-wavelengths from different sources to different detection elements of the light detector 212.

As mentioned previously, in one mode of operation, the light sources 210 are modulated and the light detector 212 are sampled by analog-to-digital converters at high speed by the high-speed drivers and analog to digital conversion circuits 132. The objective is to detect increased or decreased fluorescent intensity along with changes in the radiative lifetime of the fluorescent dye or fluorophore. The switching rates of the electronics 132 are thus often in the MegaHertz (MHz) to GigaHertz (GHz) regime or at lower frequencies if only fluorescent intensity is to be measured, or dyes with longer characteristic lifetimes are in use.

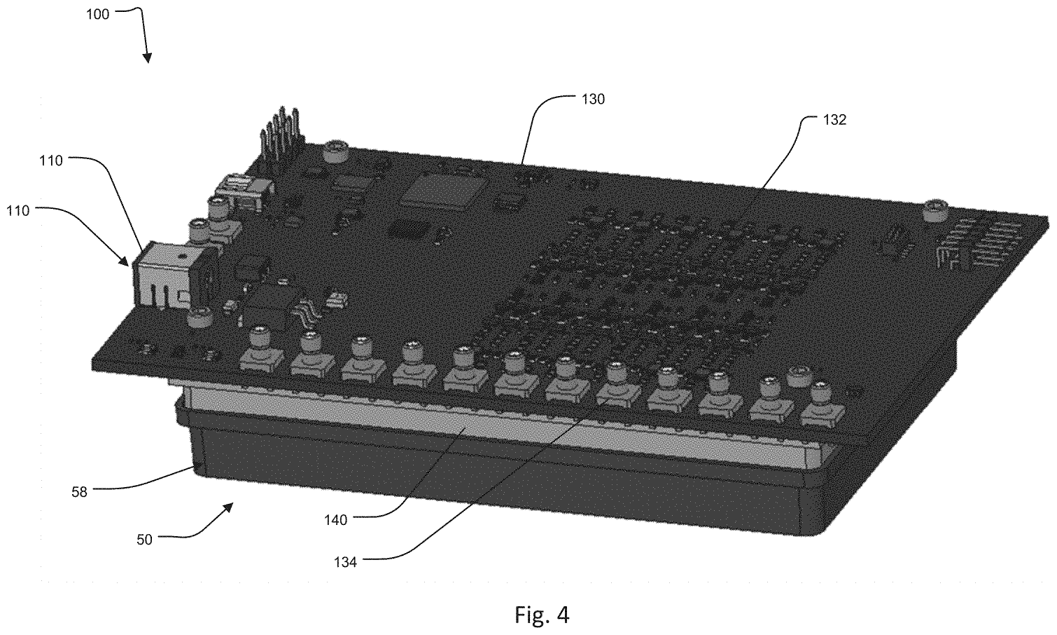

FIG. 4 is a perspective view of the well array 50 with an integrated fluorescence lifetime reader 100 attached to the well array 50, according to another embodiment of the invention.

Here the tubes 54 of the previous embodiment have been replaced with a well substrate 58.

In one embodiment, the well substrate 58 further includes a heating and cooling system for heating and cooling the samples in each of the wells. This enables the cyclic heating and cooling of the samples as is required to perform qPCR in the wells. This heating and cooling system is operated under the control of the controller 30( )of the reader on the PCB 130.

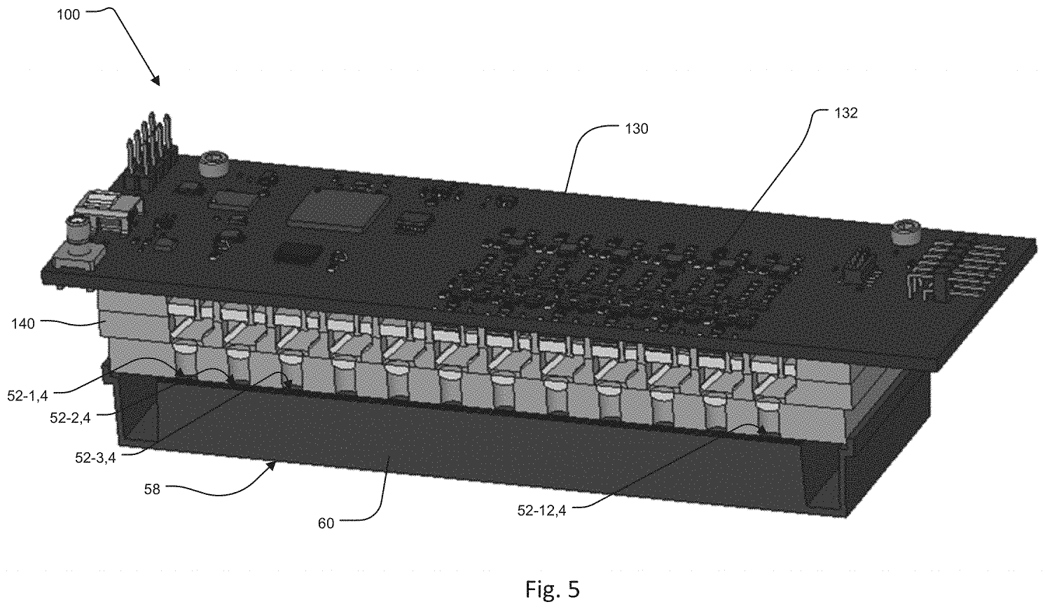

FIG. 5 is a side plan view showing the wells 52-x,y and the respective unit well subsystems 142-x,y in the optical block 140 associated with each well 52.

It shows the wells 52 are provided in the well substrate 58 along with the heating and cooling system 60 in the well substrate under the wells for heating and cooling the wells under the control of the controller 300. In one embodiment, the heating and cooling system 60 comprises a single thermoelectric cooler or multiple thermoelectric coolers for cooling the well substrate 58 and thus the samples in each of the wells 52.

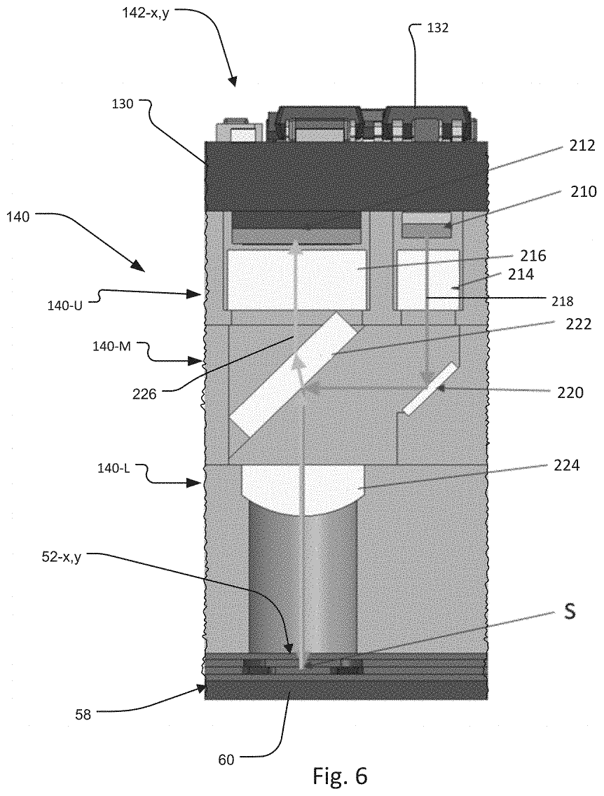

FIG. 6 is a side plan view of an exemplary single well 52-x,y and respective unit well subsystem 142-x,y associated with the well 52, according to the second embodiment.

This shows the integrated well 52 in the well substrate 58 and how the lower subblock 140-L seals against the well substrate and around each integrated well 52.

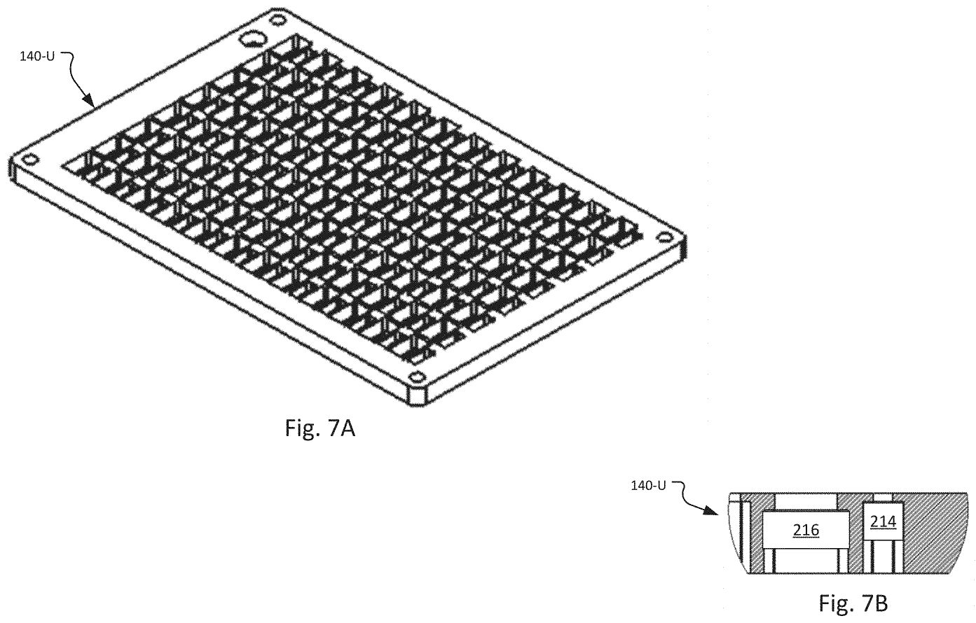

FIG. 7A is a perspective view showing the details of the upper subblock 140-L. FIG. 7B is a cross section view of a portion of the upper subblock 140-L showing the lips formed in the upper subblock 140-L for holding the filters 216, 214. Adhesive can be applied to these lips to secure the filters in place.

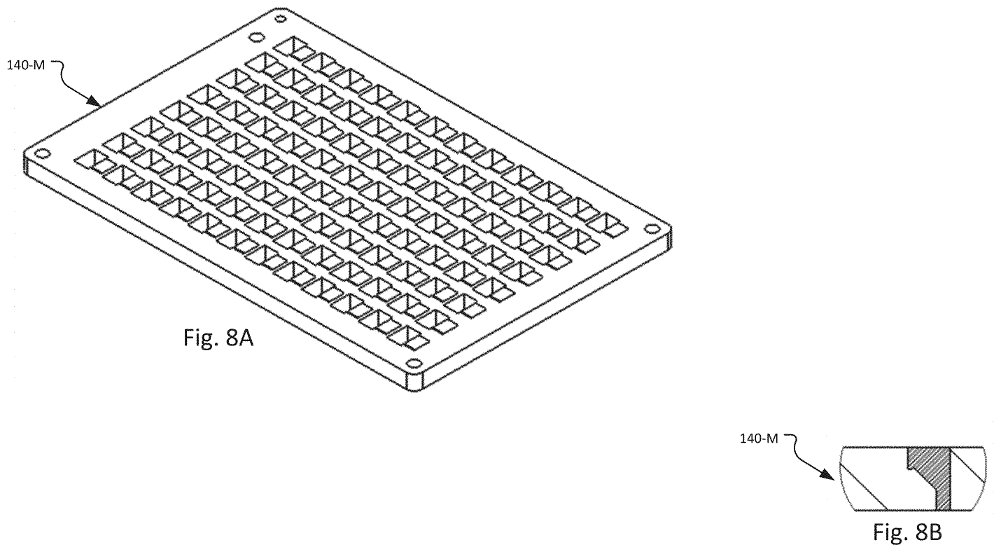

FIG. 8A is a perspective view showing the details of the middle subblock 140-M. FIG. 8B is a cross section view of a portion of the middle subblock 140-M. Two 45 degree angled brackets support the dichroic filter 222. An angled shelf supports the mirror 220. Adhesive can be applied to the lip of the bracket and to the angled shelf the secure the mirror and dichroic in place.

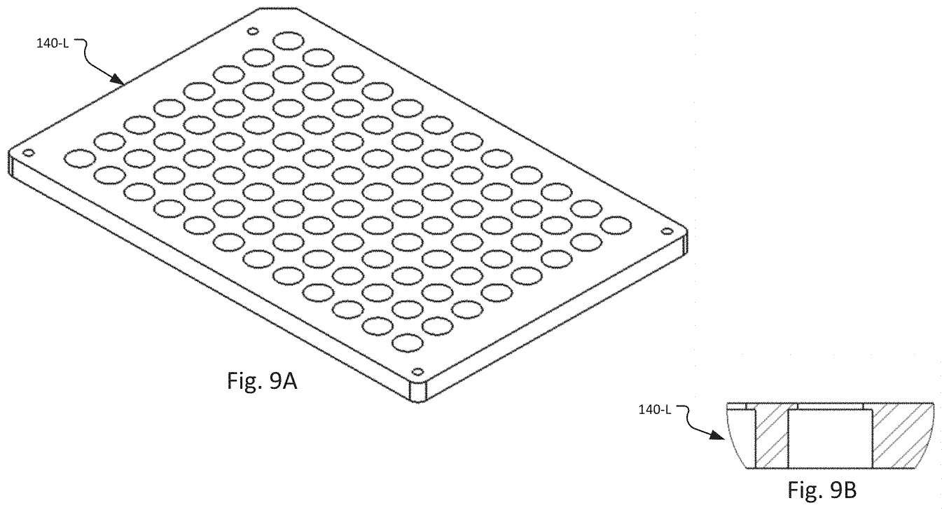

FIG. 9A is a perspective view showing the details of the lower subblock 140-L. FIG. 9B is a cross section view of a portion of the lower subblock 140-L. Here, the lens 224 is supported by a lip at the base of the subblock. Adhesive can be used to affix the lens to the fixture. In an alternative embodiment, the lens is held in place by a spring-loaded retaining ring. In yet another embodiment the lens slot is threaded and the lens is held in place by a threaded retaining ring.

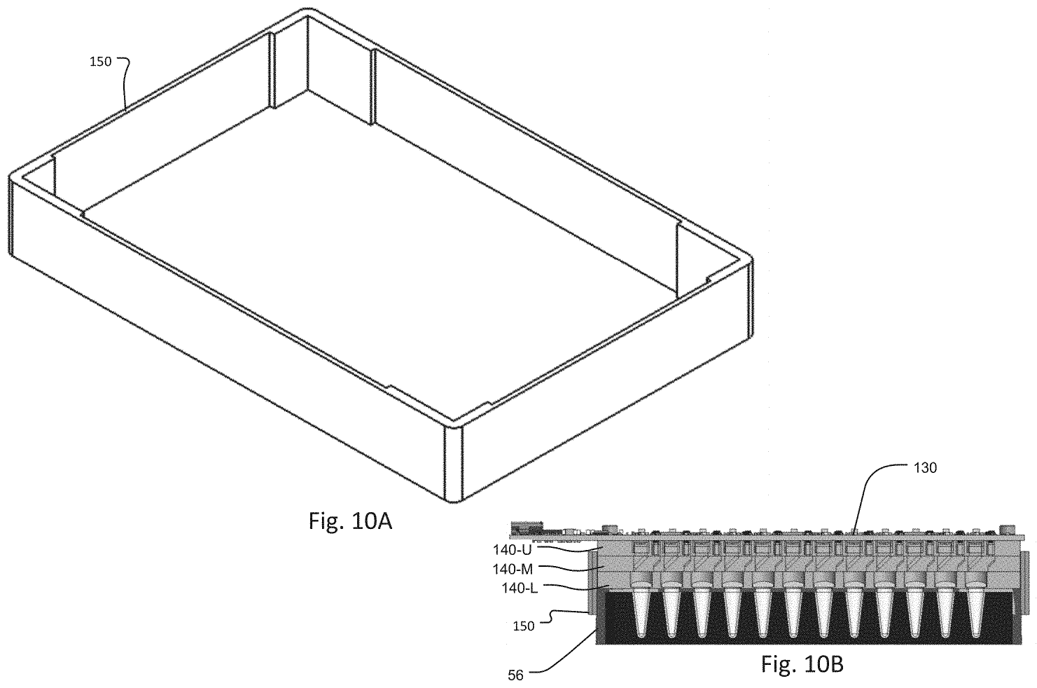

FIG. 10A is a perspective view of a skirt 150 that aligns the optical block 140 to the tube frame 56. FIG. 110B a cross sectional view showing the skirt 150 in relation to the optical block 140 and well substrate 56.

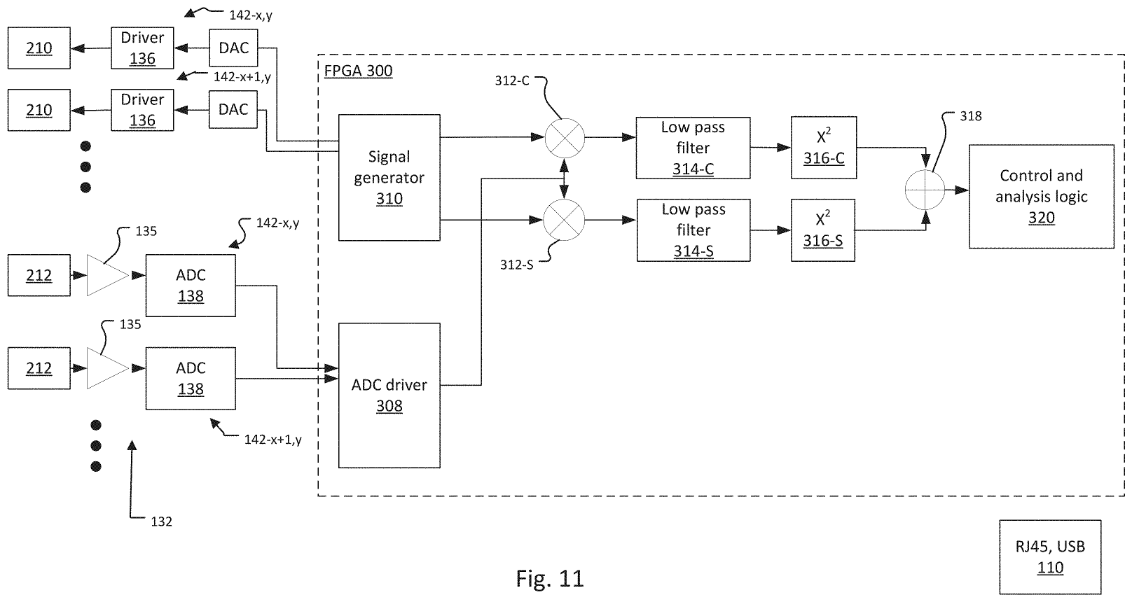

FIG. 11 is a block diagram showing details of the controller 300 and its operation of the reader 100 and specifically the unit well subsystems 142 and high-speed drivers and analog to digital conversion circuits 132 in order to perform lock-in detection.

In general, there are a number ways of implementing the controller 300 In one example, the controller 300 is a microcontroller such as a signal processing microcontroller that directs functionality of the reader 100 by executing software/firmware instructions and/or an operating system. In one example, the controller 300 is a small single-board computer. In other examples, the controller is a microcontroller unit or a system on a chip (SoC), including one or more processor cores along with memory and programmable input/output peripherals such as analog to digital convertors and digital to analog converters,

In the current implementation, the controller 300 is a Field Programmable Gate Arrays (FPGA) integrated circuit.

In the illustrated example, the FPGA controller 300 is programmed to include a signal generator 310. Often this is implemented as a look up table. In the current example, the signal generator 310 generates a reference waveform. Preferably, the signal generator 310 can generate several different types of waveforms under the control of control and analysis logic 320. These include sinusoids (cosine and sine waveforms square waves, and square waves with a selectable duty cycle.

The selected waveform is provided by the signal generator 310 through digital to analog converters (DAC) to driver circuits 136 of the high-speed drivers and analog to digital conversion circuits 132. In one example, there is a DAC and driver circuit 136 associated with each unit well subsystem 142. The driver circuits 136 provide driver currents to the one or more diodes (light emitting and/or laser) of each light source 210 associated with its respective unit well subsystem 142.

At the same time, the light detectors 212 of each of the unit well subsystems 142 are sampled. In the illustrated example, the photocurrents from one or more photodiodes of the light detectors 212 of each unit well subsystem 142 is converted into a voltage by transimpedance amplifiers 135 of the high-speed drivers and analog to digital conversion circuits 132. This voltage undergoes analog to digital conversion by analog to digital converters 138 also of the circuits 132.

The controller 300 includes two multipliers 312-C, 312-S that multiply the drive signal to the drivers 136 and phase-shifted versions of the drive signal, such as a cosine and sine wave, with each signal derived from each light detector 212 of each unit well subsystem 142. Each pipeline further includes respective low pass filters 314-C, 314-S for low pass filtering the output from the multipliers 312-C, 312-S. The output is then provided to two squaring functions 316-C, 316-S. Then the signals are summed in a summing function 318. This provides a convolution between the drive signal and the response of each light detector 212 of each unit well subsystem 142.

The control and analysis logic 320 is typically used to extract the phase difference between the drive signal and the response from each light detector along with the amplitude of the detectors' responses in order to assess the fluorescent lifetimes of the fluorophores contained in each sample S in each well 52.

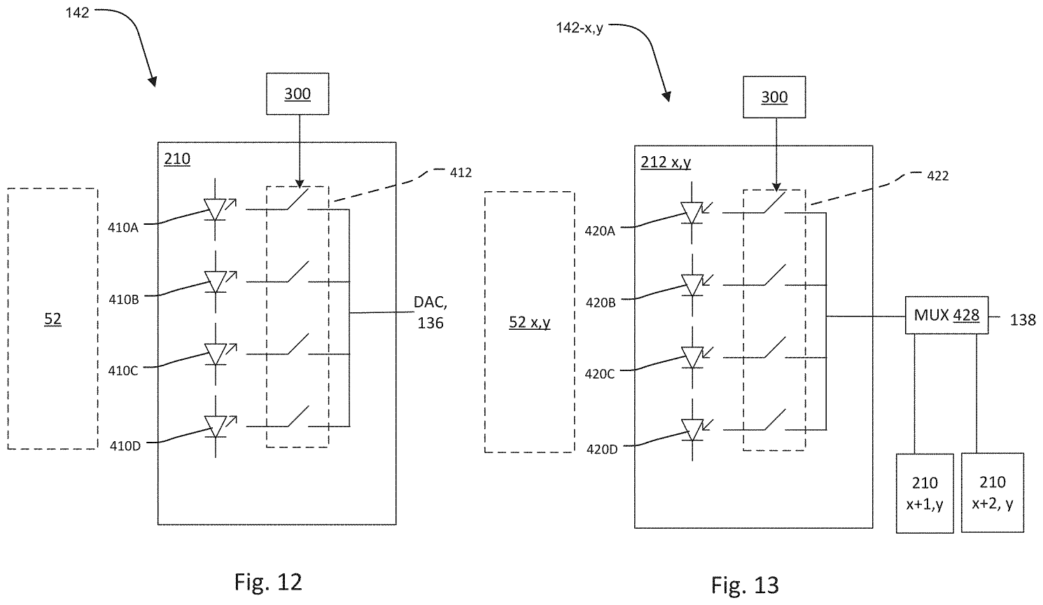

FIG. 12 is a block diagram showing the one implementation of the light source 210. In more detail, the output from the digital to analog converter DAC for an exemplary unit well subsystem 142 is provided to a driver circuit 136 and then to a switch array 412 of the light source 210. The controller 300 closes one or more of the switches to provide the drive signal to one or more of diodes 410A, 410B, 410C, 410D of the light source 210. In one example, laser diode 410A generates light for exciting a first fluorophore in the sample S in the respective well 52. Laser diode 410B generates light for exciting a second fluorophore in the sample S in the respective well 52. Diode 410C is a heating LED for raising a temperature of the sample in the respective well 52. Finally, diode 410D excites a fluorophore associated with detecting an oxygen concentration within the sample S.

FIG. 13 is a block diagram showing the one implementation of the light detector 212 and associated circuitry. In more detail, the light from the corresponding well 5 -x,y is detected by photodiodes 420A, 420B, 420C, 420D of the light detector 210 for an exemplary unit well subsystem 142. A switch array 422 connects the output of one of the photodiodes 420A, 420B, 420C, 420D to the analog to digital converter 138. The controller 300 closes one of the switches of the array 422 to provide the corresponding photodiode signal of one of diodes 420A, 420B, 420C, 420D for sampling. In one example, photodiode 420A detects photons emitted from the first fluorophore in the sample S in the respective well 52. Photodiode 420B detects photons from the second fluorophore in the sample S in the respective well 52. Microbolometer 420C detects thermal photons from the sample to assess the temperature of the sample S. Finally, photodiode 420D detects photons from the fluorophore associated with detecting an oxygen concentration within the sample S.

In the illustrated example, an analog multiplexer 428 allows the controller 300 to select a signal from only one of a group of light detectors 212 for conversion by the analog to digital converter 138. This allows one converter 138 to be shared among a group of the unit well subsystems 142 to lower the system cost.

Based on this operation, the reader 100 is useful in performing qPCR assays.

Typical qPCR assays use fluorescence amplitude to monitor the quantity of replicated DNA during the thermal cycling and polymerase chain reaction. The majority of standard dyes used in these assays also exhibit large changes in fluorescence lifetime as a result of either binding to DNA or the separation of a fluorophore and quencher molecule. Using phase-modulation, changes in fluorescence lifetime can be measured by the reader 100.

To measure DNA concentration, the samples S to be characterized are prepared as typical for qPCR assays by mixing with an appropriate buffer and an aqueous solution containing dye molecules, DNA probes and/or primers, dNTPs, dye molecules, and DNA polymerase.

In one implementation of this assay, a DNA binding dye that changes fluorescence lifetime upon binding to DNA is employed. One example dye is the commonly used, commercially available ThermoFisher Sybr Green. The Sybr Green lifetime is 4.4 nanoseconds (ns) when bound to DNA, and 3.1 picoseconds (ps) when free. The readout of this assay requires an implementation with electronics that is at GHz speed. or faster.

An alternative dye is Acridine orange, which has a fluorescence lifetime of 20-35 milliseconds (ms) when bound to DNA and 0.5 ms with no DNA present. While this dye is not commonly used for qPCR, the significantly longer fluorescence lifetimes mean that this assay can be implemented on electronics with speed in the 100 KHz range, potentially providing a lower cost hardware solution.

In an alternative implementation of this assay, a fluorophore and quencher molecule are bound to a DNA probe, for example the commonly used TaqMan assay which uses a DNA probe with a 5′ reporter dye and a 3′ quencher. First the sample dsDNA is dehybridized, and the probe molecule binds to the sample DNA. Next, the DNA polymerase copies the template strand and the fluorophore is cleaved from the probe molecule, separating the reporter dye and quencher, yielding a fluorescent signal. In one example, a fluorescein-rhodamine fluorophore-quencher pair can be used. This pair of molecules has decays of ˜4-5 ns separated and ˜ps when complexed (Hochstrasser 1992).

After the sample is loaded with the appropriate reagents into a multiwell plate, the plate is interfaced with the reader and actuator system 100 using the skirt and mounted on a thermal cycler to carry out the PCR.

The fluorescence lifetime is monitored by reader 100 by measuring the phase difference between the stimulus and fluorescence signals. The data from reader 100 is transferred to a computer, where the data from the signal for each cycle is processed and compared to standard curves to quantify target concentration in the sample.

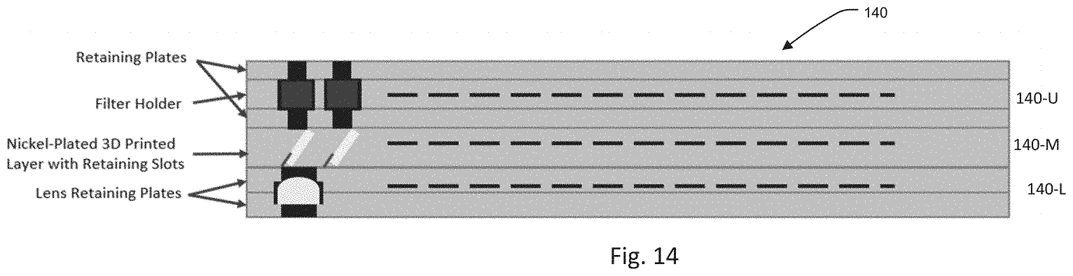

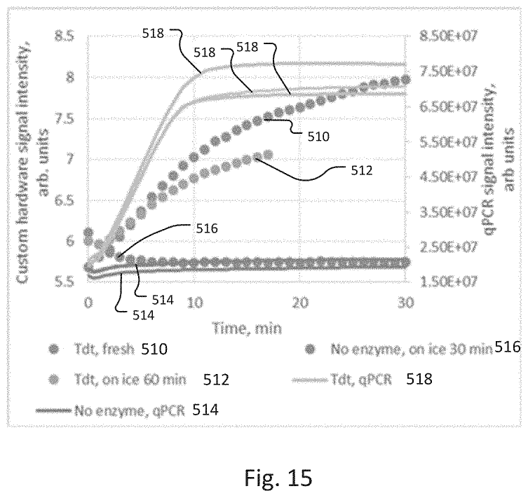

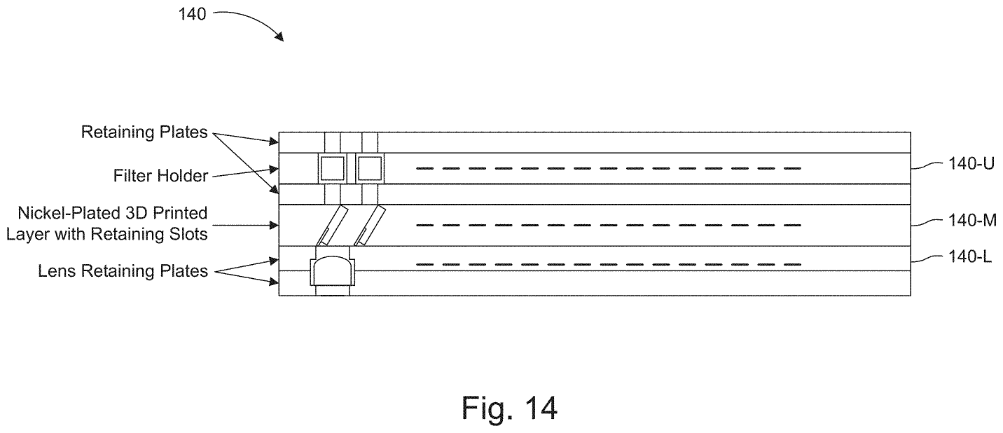

In an alternative embodiment, depicted in FIG. 14, the optical block 140 has six layers. This embodiment uses retaining plates with small clearance to optical components to ease machining and eliminate need for epoxy. In this embodiment, the upper subblock 140-U has 3 layers, with two retaining plates and a filter holder layer. The retaining plates are secured to the filter holder using recessed screws. The middle subblock 140-M is built with retaining slots to hold each dichroic filter. These inserts allow for epoxy steps to be separated from component installation, enabling component placement with a robotic pick and place tool. The lower subblock 140-L is built from two lens retaining plates that are held together with recessed screws. The three assembled subblocks are then connected using additional recessed screws.

In an alternative embodiment, the lens layer is assembled from a preformed molded lens array. In some embodiments, cuts between lens with a laser or other source may be used to prevent light leakage due to waveguiding between wells.

In an alternative embodiment the dichroic filters are formed from a single plastic sheet, such as those produced by Everix Optical Filters. An adhesive preform is applied to the plastic filter sheet, then a laser is used to cut out three sides of a rectangle. A preformed fixture is used to press the dichroic on to the angled ledge, bending the plastic filter and affixing it in place.

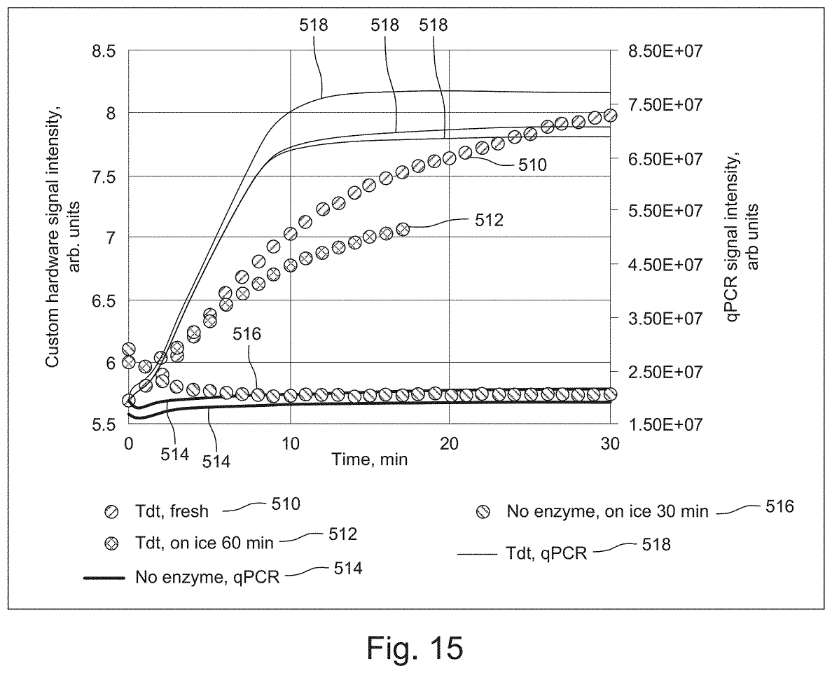

FIG. 15 is a plot of signal intensity as a function of time in minutes for fresh Tdt 510, Tdt on ice 512, a qPCR sample with no enzyme 514, a sample with no enzyme on ice 516, and qPCR with Tdt 518 as detected by the reader 100.

An alternative application of the reader 100 is the functional evaluation or screening of photo-controlled DNA polymerases or other DNA modifying enzymes, as described in PCT International Publication No. WO 2018/217689 A1, which is incorporated herein by this reference in its entirety. In this assay, the appropriate reagents (i.e. seed DNA, nucleotides, dye molecules, buffer) and candidate enzymes are populated in individual wells. The enzyme is stimulated by the reader 100 with the activation-wavelength. The well is interrogated with the excitation-wavelength and evaluated by detecting the emission-wavelength. If the enzyme is functional there will be a change in fluorescence signal due to interactions between the DNA and the dye molecule. This method can be used to characterize the function and kinetics of a photo-controlled enzyme.

An additional alternative application of the reader 100 is the measurement of oxygen. Dyes that exhibit changes in phosphorescence, as described by Gewehr, “Optical oxygen sensor based on phosphorescence lifetime quenching and employing a polymer immobilised metalloporphyrin probe,” Medical & Biological Engineering & Computing, 1993. They are commercially available in sticker form from companies such as by PreSens Precision Sensing GmbH, see Oxygen Sensor Spot SP-PSt3-NAU. These stickers can be affixed into each individual well to characterize a biochemical reaction. In one implementation, the well is a model of a human organ and contains human organ tissue, as described in U.S. Pat. Pub. No. US 2018/0142196 A1, which is incorporate herein by the reference in its entirety.

While this invention has been particularly shown and described with references to preferred embodiments thereof, it will be understood by those skilled in the art that various changes in form and details may be made therein without departing from the scope of the invention encompassed by the appended claims.

1. A well reader for a well array, comprising:
an array of detectors, each detector for detecting a fluorescence signal from fluorophores in a respective well of the well array; and
an excitation subsystem for exciting the fluorophores in the wells of the well array.

2. The reader of claim 1, wherein the well array has 96 wells.

3. The reader of claim 1, wherein the well array comprises an array of microcentrifuge tubes.

4. The reader of claim 1, wherein the well array comprises a well array substrate having an array of wells.

5. The reader of claim 1, further comprising a printed circuit board and optical block for interfacing the printed circuit board to the well array.

6. The reader of claim 5, wherein the printed circuit board includes high-speed drivers and analog to digital conversion circuits.

7. The reader of claim 5, wherein the printed circuit board includes a controller for performing a convolution between a drive signal to the excitation subsystem and a. signal produced by the detectors.

8. The reader of claim 5, wherein the printed circuit board includes a multiplexer between the array of detectors and the analog to digital conversion circuits.

9. The reader of claim 5, wherein the well reader assesses changes in a radiative lifetime of one or more fluorophores in the well array.

10. The reader of claim 5, wherein the array of detectors includes multiple photodiodes for each of the wells.

11. The reader of claim 5, wherein the excitation subsystem is implemented as a light source for each of the wells.

12. The reader of claim 11, wherein the light source includes multiple diodes for interrogating and/or heating samples held in the respective well of the well array.

13. A method of operation of a well reader, comprising:
exciting fluorophores in wells of the well array; and
detecting a fluorescence signal from one or more fluorophores in the wells of the well array with an array of detectors.

14. The method of claim 13, wherein there is a respective detector for each of the wells.

15. The method of claim 13, further comprising assessing changes in a radiative lifetime of the one or more fluorophores.

16. A well reader and actuator for a well array, comprising:
an array of unit well subsystems for the well array; and
a controller for controlling monitoring polymerase chain reactions in the wells via the unit well subsystems.

17. A method of DNA quantification that uses optical phase modulation to measure changes in fluorescence lifetime of DNA binding dyes during polymerase chain reactions taking place in a well array.

18. A reaction system, comprising:
a well array; and
a fluorescence lifetime analysis system for assessing changes in the radiative lifetime of fluorophores in the well array.
